(19) United States
(12) Patent Application Publication
(10) Pub. No.: US 2007/0050924 A1
(43) Pub. Date: Mar. 8, 2007

(54) Composition for the oxidation dyeing of keratin fibres, comprising at least one 4,5- or 3,4-diaminopyrazole or a triaminopyrazole and at least one particular cellulose-based compound, and dyeing process

(75) Inventor: Jean Cotteret, Verneuil Sur Seine (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Clark, NJ (US)

(21) Appl. No.: 11/595,089

(22) Filed: Nov. 9, 2006

Related U.S. Application Data
(63) Continuation of application No. 10/451,647, filed on Nov. 25, 2003, now abandoned.

(30) Foreign Application Priority Data
Dec. 22, 2000 (FR) 00/16953

(51) Int. Cl. A61K 8/00

(52) U.S. Cl. 8/405

(57) ABSTRACT

The present invention relates to a composition for the oxidation dyeing of keratin fibres, in particular of human keratin fibres such as the hair, comprising at least one oxidation base chosen from 4,5- or 3,4-diaminopyrazoles and triaminopyrazoles, in combination with at least one particular cellulose-based compound, and also to the dyeing process using this composition with an oxidizing agent.

### CROSS REFERENCE TO RELATED APPLICATIONS

The present application is a continuation of U.S. application Ser. No. 10/451,647, filed Nov. 25, 2003, which is is a national phase entry under 35 U.S.C. §371 of International Application PCT/FR0103728, filed Nov. 26, 2001 which claims priority from French Application No. 00/16953, filed Dec. 22, 2000, the disclosures of which are hereby incorporated by reference herein.

### BACKGROUND OF THE INVENTION

The present invention relates to a composition for the oxidation dyeing of keratin fibres, in particular of human keratin fibres such as the hair, and also to the dyeing process using this composition with an oxidizing agent.

It is known practice to dye keratin fibres, and in particular human hair, with dye compositions containing oxidation dye precursors, in particular ortho- or para-phenylenediamines, ortho- or para-aminophenols or heterocyclic compounds such as pyrazole derivatives which are generally referred to as oxidation bases. Oxidation dye precursors, or oxidation bases, are colourless or weakly coloured compounds which, when combined with oxidizing products, can give rise to coloured compounds and dyes by a process of oxidative condensation.

It is also known that the shades obtained with these oxidation bases can be varied by combining them with suitably selected couplers or coloration modifiers, the latter being chosen in particular from aromatic meta-diamines, meta-aminophenols, meta-diphenols and certain heterocyclic compounds.

The variety of molecules used as regards the oxidation bases and the couplers allows a wide range of colours to be obtained.

The “permanent” coloration obtained by means of these oxidation dyes must moreover satisfy a certain number of requirements. Thus it must not have any toxicological disadvantages, it must be able to give shades of the desired intensity and it must be able to withstand external agents (light, bad weather, washing, permanent-waving, perspiration, rubbing).

The dyes must also be able to cover white hair and, lastly, they must be as unselective as possible, i. e. they must give the smallest possible colour differences along the same length of keratin fibre, which may in fact be differently sensitized (i.e. damaged) between its end and its root.

Compositions for the oxidation dyeing of keratin fibres, containing pyrazole derivatives such as 4,5-diaminopyrazoles, 3,4-diaminopyrazoles or 3,4,5-triaminopyrazoles as oxidation base have already been proposed, especially in German patent applications DE 3 843 892, DE 4 234 887, DE 4 234 886, DE 4 234 885 or DE 195 43 988. However, such compositions are not entirely satisfactory since, during the dyeing processes, side reactions take place that can have adverse effects in terms of the harmlessness and of the dyeing properties obtained, and especially the strength and resistance of the colorations with respect to the various attacking factors to which the hair may be subjected.

The aim of the invention is to develop novel dye compositions that do not have the drawbacks of the dyes of the prior art, in particular strong dyes that are particularly resistant to the various attacking factors to which the hair may be subjected, and that show good harmlessness.

To this end, one subject of the invention is a composition for the oxidation dyeing of human keratin fibres, and in particular of human keratin fibres such as the hair, characterized in that it comprises, in a medium that is suitable for dyeing:
at least one oxidation base chosen from 4,5- or 3,4-diaminopyrazoles and triaminopyrazoles, and
at least one cellulose-based compound chosen from unsubstituted celluloses and cellulose ethers; with the proviso that when the said cellulose-based compound is a hydroxyethylcellulose, then the said oxidation base is other than 3,4-diaminopyrazole, 4,5-diamino-1-methylpyrazole and 4,5-diamino-1-(4′-methoxybenzyl)pyrazole.

The oxidation dye composition of the invention makes it possible to obtain, with good harmlessness, strong, relatively unselective colorations in varied shades, which show excellent resistance properties both with respect to atmospheric agents such as light and bad weather and with respect to perspiration and various treatments to which the hair may be subjected (shampooing or permanent reshaping).

Except where otherwise mentioned, all the radicals, substituents, groups and chains in the context of the invention are linear or branched, and substituted or unsubstituted.

According to the invention, “cellulose-based compound” means any polysaccharide compound containing in its structure sequences of glucose residues linked via β-1,4 bonds. Besides unsubstituted celluloses, the cellulose derivatives may be anionic, cationic, amphoteric or nonionic. Among these derivatives that may be distinguished are cellulose ethers, cellulose esters and cellulose ester ethers. Among the cellulose esters are mineral esters of cellulose (cellulose nitrates, sulphates, phosphates, etc.), organic esters of cellulose (cellulose monoacetates, triacetates, amidopropionates, acetate butyrates, acetate propionates, acetate trimellitates, etc.) and mixed organic/mineral esters of cellulose such as cellulose acetate butyrate sulphates and cellulose acetate propionate sulphates. Among the cellulose ester ethers that may be mentioned are hydroxypropylmethylcellulose phthalates and ethylcellulose sulphates.

The cellulose-based compounds of the invention are exclusively chosen from unsubstituted celluloses, including those in a microcrystalline form, and cellulose ethers.

Among the nonionic cellulose ethers that may be mentioned are alkylcelluloses such as methylcelluloses and ethylcelluloses; hydroxyalkylcelluloses such as hydroxymethylcelluloses, hydroxyethylcelluloses and hydroxypropylcelluloses; mixed hydroxyalkyl-alkylcelluloses such as hydroxypropyl-methylcelluloses, hydroxyethyl-methylcelluloses, hydroxyethyl-ethylcelluloses and hydroxybutyl-methylcelluloses.

Among the anionic cellulose ethers that may be mentioned are carboxyalkylcelluloses and salts thereof. Examples that may be mentioned include carboxymethylcelluloses, carboxymethylmethylcelluloses and carboxymethylhydroxyethylcelluloses, and the sodium salts thereof.

Among the cationic cellulose ethers that may be mentioned are crosslinked or non-crosslinked quaternized hydroxyethylcelluloses. The quaternizing agent may especially be glycidyltrimethylammonium chloride or a fatty amine such as laurylamine or stearylamine. Another cationic cellulose ether that may be mentioned is hydroxyethylcellulosehydroxypropyl-trimethylammonium.

The cellulose-based compound(s) in accordance with the invention preferably represent(s) from 0.00001% to 10% by weight approximately relative to the total weight of the dye composition, even more preferably from 0.001% to 5% and even more preferentially from 0.001% to 3% by weight approximately relative to this weight.

One particular form of the invention consists of a composition for the oxidation dyeing of human keratin fibres, and in particular of human keratin fibres such as the hair, characterized in that it comprises, in a medium that is suitable for dyeing:
at least one oxidation base chosen from 4,5- or 3,4-diaminopyrazoles and triaminopyrazoles,
at least one cellulose-based compound chosen from unsubstituted celluloses; anionic or cationic cellulose ethers; nonionic cellulose ethers chosen from alkylcelluloses, hydroxymethylcelluloses, hydroxypropylcelluloses and hydroxyalkyl-alkylcelluloses.

Among the 4,5- or 3,4-diaminopyrazoles that are useful in the dye compositions of the invention, mention may be made particularly of the diaminopyrazoles chosen from the 4,5- or 3,4-diaminopyrazoles of formula (I) or (II) below, and/or the addition salts thereof with an acid: 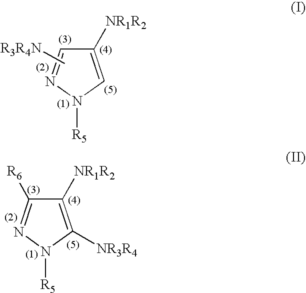
in which:
R₁, R₂, R₃, R₄ and R₅, which may be identical or different, represent a hydrogen atom; a C₁-C₆ alkyl radical which is unsubstituted or substituted with at least one substituent chosen from OR, NHR, NRR′, SR, SOR, SO₂R, COR, COOH, CONH₂, CONHR, CONRR′, PO(OH)₂, SH and SO₃X, a non-cationic heterocycle, Cl, Br or I, X denoting a hydrogen atom, Na, K or NH₄, and R and R′, which may be identical or different, representing a C₁-C₄ alkyl or alkenyl; a C₂-C₄ hydroxyalkyl radical; a C₂-C₄ aminoalkyl radical; a phenyl radical; a phenyl radical substituted with a halogen atom or a C₁-C₄ alkyl, C₁-C₄ alkoxy, nitro, trifluoromethyl, amino or C₁-C₄ alkylamino radical; a benzyl radical; a benzyl radical substituted with a halogen atom or with a C₁-C₄ alkyl, C₁-C₄ alkoxy, methylenedioxy or amino radical; 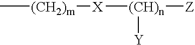
   a radical
in which m and n are integers, which may be identical or different, between 0 and 3 inclusive, X represents an oxygen atom or an NH group, Y represents a hydrogen atom or a C₁-C₄ alkyl radical, and Z represents a methyl radical when n is equal to 0, or Z represents a C₁-C₄ alkyl radical, a group OR or NR″R′″ when n is greater than or equal to 1, R″ and R′″, which may be identical or different, denoting a hydrogen atom or a C₁-C₄ alkyl radical; or R₉ forms with the nitrogen atom of the group NR₇R₈ in position 5 an at least 4-membered heterocycle,
R₆ represents a C₁-C₆ alkyl radical; a C₁-C₄ hydroxyalkyl radical; a C₁-C₄ aminoalkyl radical; a (C₁-C₄)alkylamino(C₁-C ₄)alkyl radical; a di (C₁-C₄)alkylamino(C₁-C₄)alkyl radical; a hydroxy (C₁-C₄) alkylamino (C₁-C₄) alkyl radical; a (C₁-C₄)alkoxymethyl radical; a phenyl radical; a phenyl radical substituted with a halogen atom or with a C₁-C₄ alkyl, C₁-C₄ alkoxy, nitro, trifluoromethyl, amino or C₁-C₄ alkylamino radical; a benzyl radical; a benzyl radical substituted with a halogen atom or with a C₁-C₄ alkyl, C₁-C₄ alkoxy, nitro, trifluoromethyl, amino or C₁-C₄ alkylamino radical; a heterocycle chosen from thiophene, furan and pyridine, or a radical —(CH₂)ₚ—O—(CH₂)_{q}—OR″, in which p and q are integers, which may be identical or different, between 1 and 3 inclusive and R″ is as defined above, it being understood that:
   at least one of the radicals R₁, R₂, R₃ and R₄ represents a hydrogen atom.

Among the triaminopyrazoles that are useful as oxidation bases in the dye compositions in accordance with the invention, mention may be made more particularly of the compounds of formula (III) below, and the addition salts thereof with an acid: 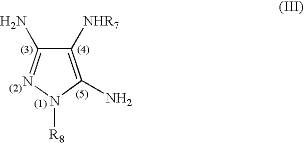
in which:
R₇ and R₈, which may be identical or different, represent a hydrogen atom or a C₁-C₄ alkyl or C₂-C₄ hydroxyalkyl radical.

Among the 4,5- or 3,4-diaminopyrazoles of formula (I) above that may be mentioned more particularly are 4,5-diamino-1- (4′-methoxybenzyl)pyrazole, 4,5-diamino-1-(4′-methylbenzyl) pyrazole, 4,5-diamino-1-(4′-chlorobenzyl)pyrazole, 4,5-diamino-1- (3′-methoxybenzyl)pyrazole, 4-amino-l- (4′-methoxybenzyl) -5-methylaminopyrazole, 4-amino-5-(□-hydroxy-ethyl) amino-1- (4′-methoxybenzyl)pyrazole, 4-amino-5-(β-hydroxyethyl) amino-1-methylpyrazole, 4-amino-(3)-5-methylaminopyrazole, 3- (5),4-diaminopyrazole, 4,5-diamino-1-methylpyrazole, 4,5-diamino-1-ethylpyrazole, 4,5-diamino-1-isopropylpyrazole, 4,5-diamino-1-hydroxyethylpyrazole, 4,5-diamino-1-benzylpyrazole, 4-diamino-5-hydroxyethylamino-1-hydroxyethylpyrazole, 4-diamino-5-methylamino-1-hydroxyethylpyrazole, 3-amino-4,5,7,8-tetrahydropyrazolo[1,5-a]pyrimidine, 7-amino-2,3-dihydro-1H-imidazole[1,2-b]pyrazole, 3-amino-8-methyl-4,5,7,8-tetrahydropyrazolo[1,5-a]pyrimidine, 2-(4,5-diamino-1-pyrazolyl)-1-ethanesulphonic acid, 2-(4,5-diamino-1-pyrazolyl)acetamide, 2-(4,5-diamino-1-pyrazolyl)acetic acid, 2-(2-dimethylaminoethyl)-2H-pyrazole-3,4-diamine and 2-(2-methoxyethyl)-2H-pyrazole-3,4-diamine, and the addition salts thereof with an acid.

The diaminopyrazoles that are useful in the present invention may be obtained via synthetic processes that are well known to those skilled in the art. For example, the 4,5-diaminopyrazoles of formula (II) may be prepared according to the synthetic process as described, for example, in French patent application FR-A-2 733 749.

Among the 4,5-diaminopyrazoles of formula (II) above that may be mentioned more particularly are:
1-benzyl-4,5-diamino-3-methylpyrazole,
4,5-diamino-l- (β-hydroxyethyl) -3- (4′-methoxyphenyl) -pyrazole,
4,5-diamino-l- (β-hydroxyethyl) -3- (4′-methylphenyl) -pyrazole,
4,5-diamino-1- (β-hydroxyethyl) -3- (3′-methylphenyl) -pyrazole,
4,5-diamino-3-methyl-1-isopropylpyrazole,
4,5-diamino-3-(4′-methoxyphenyl)-1-isopropylpyrazole,
4,5-diamino-1-ethyl-3-methylpyrazole,
4,5-diamino-1-ethyl-3- (4′-methoxyphenyl)pyrazole,
4,5-diamino-3-hydroxymethyl-1-methylpyrazole,
4,5-diamino-1-ethyl-3-hydroxymethylpyrazole,
4,5-diamino-3-hydroxymethyl-1-isopropylpyrazole,
4,5-diamino-3-hydroxymethyl-1-tert-butylpyrazole,
4,5-diamino-3-hydroxymethyl-1-phenylpyrazole,
4,5-diamino-3-hydroxymethyl-1- (2′-methoxyphenyl)pyrazole,
4,5-diamino-3-hydroxymethyl-1- (3′-methoxyphenyl)pyrazole,
4,5-diamino-3-hydroxymethyl-1- (4′-methoxyphenyl)pyrazole,
1-benzyl-4,5-diamino-3-hydroxymethylpyrazole,
4,5-diamino-3-methyl-1- (2′-methoxyphenyl)pyrazole,
4,5-diamino-3-methyl-1- (3′-methoxyphenyl)pyrazole,
4,5-diamino-3-methyl-1- (4′-methoxyphenyl)pyrazole,
3-aminomethyl-4,5-diamino-1-methylpyrazole,
3-aminomethyl-4,5-diamino-1-ethylpyrazole,
3-aminomethyl-4,5-diamino-1-isopropylpyrazole,
3-aminomethyl-4,5-diamino-1-tert-butylpyrazole,
4,5-diamino-3-dimethylaminomethyl-1-methylpyrazole,
4,5-diamino-3-dimethylaminomethyl-1-ethylpyrazole,
4,5-diamino-3-dimethylaminomethyl-1-isopropylpyrazole,
4,5-diamino-3-dimethylaminomethyl-1-tert-butylpyrazole,
4,5-diamino-3-ethylaminomethyl-1-methylpyrazole,
4,5-diamino-3-ethylaminomethyl-1-ethylpyrazole,
4,5-diamino-3-ethylaminomethyl-1-isopropylpyrazole,
4,5-diamino-3-ethylaminomethyl-1-tert-butylpyrazole,
4,5-diamino-3-methylaminomethyl-1-methylpyrazole,
4,5-diamino-3-methylaminomethyl-1-isopropylpyrazole,
4,5-diamino-1-ethyl-3-methylaminomethylpyrazole,
1-tert-butyl-4,5-diamino-3-methylaminomethylpyrazole,
4,5-diamino-3-[(β-hydroxyethyl)aminomethyl]-1-methyl-pyrazole,
4,5-diamino-3-[(β-hydroxyethyl)aminomethyl]-1-iso-propylpyrazole,
4,5-diamino-1-ethyl-3-[(β-hydroxyethyl)aminomethyl]-pyrazole,
1-tert-butyl-4,5-diamino-3-[(β-hydroxyethyl)amino-methyl]pyrazole,
4-amino-5-(β-hydroxyethyl)amino-1,3-dimethylpyrazole,
4-amino-5-(β-hydroxyethyl)amino-1-isopropyl-3-methyl-pyrazole,
4-amino-5-(β-hydroxyethyl)amino-1-ethyl-3-methylpyrazole,
4-amino-5-(β-hydroxyethyl)amino-1-tert-butyl-3-methylpyrazole,
4-amino-5-(β-hydroxyethyl)amino-1-phenyl-3-methylpyrazole,
4-amino-5-(β-hydroxyethyl)amino-1-(2-methoxyphenyl)-3-methylpyrazole,
4-amino-5-(β-hydroxyethyl)amino-1-(3-methoxyphenyl)-3-methylpyrazole,
4-amino-5-(β-hydroxyethyl)amino-1-(4-methoxyphenyl)-3-methylpyrazole,
4-amino-5-(β-hydroxyethyl)amino-1-benzyl-3-methylpyrazole,
4-amino-1-ethyl-3-methyl-5-methylaminopyrazole,
4-amino-1-tert-butyl-3-methyl-5-methylaminopyrazole,
4,5-diamino-1,3-dimethylpyrazole,
4,5-diamino-3-tert-butyl-1-methylpyrazole,
4,5-diamino-1-tert-butyl-3-methylpyrazole,
4,5-diamino-1-methyl-3-phenylpyrazole,
4,5-diamino-1-(β-hydroxyethyl) -3-methylpyrazole,
4,5-diamino-1-(β-hydroxyethyl)-3-phenylpyrazole,
4,5-diamino-1-methyl-3- (2′-chlorophenyl)pyrazole,
4,5-diamino-1-methyl-3- (4′-chlorophenyl)pyrazole,
4,5-diamino-1-methyl-3- (3′-trifluoromethylphenyl)pyrazole,
4,5-diamino-1,3-diphenylpyrazole,
4,5-diamino-3-methyl-1-phenylpyrazole,
4-amino-1,3-dimethyl-5-phenylaminopyrazole,
4-amino-1-ethyl-3-methyl-5-phenylaminopyrazole,
4-amino-1,3-dimethyl-5-methylaminopyrazole,
4-amino-3-methyl-1-isopropyl-5-methylaminopyrazole,
4-amino-3-isobutoxymethyl-1-methyl-5-methylaminopyrazole,
4-amino-3-methoxyethoxymethyl-1-methyl-5-methylamino-pyrazole,
4-amino-3-hydroxymethyl-1-methyl-5-methylaminopyrazole,
4-amino-1,3-diphenyl-5-phenylaminopyrazole,
4-amino-3-methyl-5-methylamino-1-phenylpyrazole,
4-amino-1,3-dimethyl-5-hydrazinopyrazole,
5-amino-3-methyl-4-methylamino-1-phenylpyrazole,
5-amino-1-methyl-4-(N,N-methylphenyl)amino-3-(4(-chlorophenyl)pyrazole,
5-amino-3-ethyl-1-methyl-4-(N,N-methylphenyl)amino-pyrazole,
5-amino-1-methyl-4-(N,N-methylphenyl)amino-3-phenyl-pyrazole,
5-amino-3-ethyl-4-(N,N-methylphenyl)aminopyrazole,
5-amino-4-(N,N-methylphenyl)amino-3-phenylpyrazole,
5-amino-4-(N,N-methylphenyl)amino-3-(4(-methylphenyl)-pyrazole,
5-amino-3-(4(-chlorophenyl)-4-(N,N-methylphenyl)-aminopyrazole,
5-amino-3-(4(-methoxyphenyl)-4-(N,N-methylphenyl)-aminopyrazole,
4-amino-5-methylamino-3-phenylpyrazole,
4-amino-5-ethylamino-3-phenylpyrazole,
4-amino-5-ethylamino-3-(4(-methylphenyl)pyrazole,
4-amino-3-phenyl-5-propylaminopyrazole,
4-amino-5-butylamino-3-phenylpyrazole,
4-amino-3-phenyl-5-phenylaminopyrazole,
4-amino-5-benzylamino-3-phenylpyrazole,
4-amino-5-(4(-chlorophenyl)amino-3-phenylpyrazole,
4-amino-3-(4(-chlorophenyl)-5-phenylaminopyrazole,
4-amino-3-(4(-methoxyphenyl)-5-phenylaminopyrazole,
1-(4′-chlorobenzyl)-4,5-diamino-3-methylpyrazole,
4,5-diamino-3-hydroxymethyl-1-isopropylpyrazole,
4-amino-1-ethyl-3-methyl-5-methylaminopyrazole,
4-amino-5-(2°-aminoethyl)amino-1,3-dimethylpyrazole, and the addition salts thereof with an acid.

Among the 4,5- or 3,4-diaminopyrazoles of formula (I) above, the following are more particularly preferred:
4,5-diamino-1-benzylpyrazole,
4,5-diamino-1-(4′-chlorobenzyl)pyrazole,
4,5-diamino-1-methylpyrazole,
4,5-diamino-1-hydroxyethylpyrazole,
2-(2-methoxyethyl)-2H-pyrazole-3,4-diamine and the addition salts thereof with an acid.

Among the 4,5-diaminopyrazoles of formula (II) above, the following are more particularly preferred:
4,5-diamino-1,3-dimethylpyrazole,
4,5-diamino-3-methyl-1-phenylpyrazole,
4,5-diamino-1-methyl-3-phenylpyrazole,
4-amino-1,3-dimethyl-5-hydrazinopyrazole,
1-benzyl-4,5-diamino-3-methylpyrazole,
4,5-diamino-3-tert-butyl-1-methylpyrazole,
4,5-diamino-1-tert-butyl-3-methylpyrazole,
4,5-diamino-1-(β-hydroxyethyl)-3-methylpyrazole,
4,5-diamino-1-ethyl-3-methylpyrazole,
4,5-diamino-1-ethyl-3-(4′-methoxyphenyl)pyrazole,
4,5-diamino-1-ethyl-3-hydroxymethylpyrazole,
4,5-diamino-3-hydroxymethyl-1-methylpyrazole,
4,5-diamino-3-hydroxymethyl-1-isopropylpyrazole,
4,5-diamino-3-methyl-1-isopropylpyrazole,
4-amino-5-(2′-aminoethyl)amino-1,3-dimethylpyrazole, and the addition salts thereof with an acid.

Among the triaminopyrazoles of formula (III) above that may be mentioned more particularly are 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

Among the 3,4- and 4,5-diaminopyrazoles and triaminopyrazoles of the invention, 4,5-diamino-1-hydroxyethylpyrazole is even more particularly preferred.

Another form of the invention that is even more particularly preferred is a composition for the oxidation dyeing of human keratin fibres, and in particular of human keratin fibres such as the hair, characterized in that it comprises, in a medium that is suitable for dyeing:
as oxidation base, at least 4,5-diamino-1-hydroxy-ethylpyrazole or an addition salt thereof, and
at least one hydroxyethylcellulose.

The 4,5- or 3,4-diaminopyrazole(s) and/or the triaminopyrazole(s) in accordance with the invention and/or the corresponding addition salt(s) with an acid preferably represent from 0.0005% to 12% by weight approximately relative to the total weight of the dye composition and more preferably from 0.005% to 6% by weight approximately relative to this weight.

Preferably, the weight ratio of the cellulose-based compound(s) to the 4,5- or 3,4-diaminopyrazole(s) and/or the triaminopyrazole(s) and/or the addition salt(s) with an acid is between 0.001 and 100 and even more preferably between 0.01 and 10.

The dye compositions in accordance with the invention preferably contain at least one coupler. The couplers that may be used are those conventionally used for oxidation dyeing, and especially meta-phenylenediamines, meta-aminophenols, meta-diphenols, naphthol derivatives and heterocyclic couplers.

The meta-phenylenediamines, meta-aminophenols and meta-diphenols which may be used as additional couplers in the dye composition in accordance with the invention are preferably chosen from the compounds corresponding to formula (1) below, and the addition salts thereof with an acid: 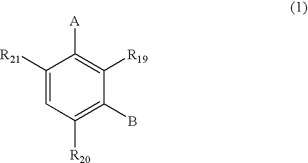
in which:
A and B, which may be identical or different, represent a hydroxyl, amino or -NHR22 radical in which R₂₂ represents a C₁-C₄ alkyl, C₁-C₄ monohydroxyalkyl or C₂-C₄ polyhydroxyalkyl radical,
R₁₉, R₂₀ and R₂₁, which may be identical or different, represent a hydrogen atom or a halogen atom such as a bromine, chlorine, iodine or fluorine atom, or a C₁-C₄ alkyl, C₁-C₄ alkoxy, C₁-C₄ monohydroxyalkyl, C₂-C₄ polyhydroxyalkyl, C₁-C₄ monohydroxyalkoxy or C₂-C₄ polyhydroxyalkoxy radical. Among the compounds of formula (1) above, mention may be made in particular of 2-methyl-5-aminophenol, 2-methyl-5-amino-6-chlorophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-((-hydroxyethyloxy)benzene, 2-amino-4-((-hydroxyethylamino)-1-methoxybenzene, 1,3-diaminobenzene and 2,6-bis(β-hydroxyethylamino)toluene, and the addition salts thereof with an acid.

The heterocyclic coupler(s) which may be used as additional couplers in the dye composition in accordance with the invention can be chosen in particular from indole derivatives, indoline derivatives, pyridine derivatives, pyrimidine derivatives and pyrazolones, and the addition salts thereof with an acid.

Among these heterocyclic couplers, mention may be made in particular, for example, of sesamol, 1-N-(β-hydroxyethyl)amino-3′, 4-methylenedioxybenzene, 6-hydroxyindole, 4-hydroxyindole, 4-hydroxy-N-methylindole, 6-hydroxyindoline, 6-hydroxybenzomorpholine, 2,6-dihydroxy-4-methylpyridine, 3,5-diamino-2,6-dimethoxypyridine, 2-amino-3-hydroxypyridine, 1-H-3-methylpyrazol-5-one and 1-phenyl-3-methylpyrazol-5-one, and the addition salts thereof with an acid.

Among the naphthol derivatives that may be mentioned are α-naphthol and 2-methyl-1-naphthol.

The additional coupler(s) preferably represent(s) from 0.0001% to 10% by weight approximately relative to the total weight of the dye composition, and even more preferably from 0.005% to 5% by weight approximately relative to this weight.

The dye compositions in accordance with the invention may also contain other oxidation bases conventionally used for oxidation dyeing, other than a diaminopyrazole and a triaminopyrazole and/or direct dyes, especially to modify the shades or to enrich them with glints.

The additional oxidation bases that may be used in the context of the present invention are chosen from those conventionally known in oxidation dyeing, and among which mention may be made especially of ortho- and para-phenylenediamines, double bases, ortho- and para-aminophenols, and heterocyclic bases other than the pyrazoles of the invention, and also the addition salts thereof with an acid, and especially:
(I) the para-phenylenediamines of formula (2) below, and the addition salts thereof with an acid: 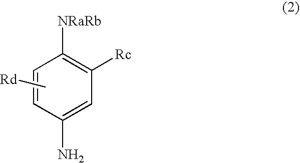
   in which:
Rₐ represents a hydrogen atom, a C₁-C₄ alkyl radical, a C₁-C₄ monohydroxyalkyl radical, a C₂-C₄ polyhydroxyalkyl radical, a (C₁-C₄)alkoxy(Cl-C₄)alkyl radical, a C₁-C₄ alkyl radical substituted with a nitrogenous group, a phenyl radical or a 4′-aminophenyl radical;
R_{b} represents a hydrogen atom, a C₁-C₄ alkyl radical, a C₁-C4 monohydroxyalkyl radical, a C₂-C₄ polyhydroxyalkyl radical, a (C₁-C₄)alkoxy(C₁-C₄)alkyl radical or a C₁-C4 alkyl radical substituted with a nitrogenous group;
R_{c} represents a hydrogen atom, a halogen atom such as a chlorine atom, a C₁-C₄ alkyl radical, a sulfo radical, a carboxyl radical, a C₁-C₄ monohydroxyalkyl radical, a C₁-C₄ hydroxyalkoxy radical, an acetylamino(C₁-C₄)alkoxy radical, a C₁-C₄ mesylaminoalkoxy radical or a carbamoylamino (C₁-C₄) alkoxy radical,
R_{d} represents a hydrogen or halogen atom or a C₁-C₄ alkyl radical,
Rₐ and R_{b} may also form with the nitrogen atom that bears them a 5- or 6-membered nitrogenous heterocycle optionally substituted with one or more alkyl, hydroxyl or ureido groups.

Among the nitrogenous groups of formula (2) above, mention may be made in particular of amino, mono(C₁-C₄) alkylamino, di (C₁-C₄)alkylamino, tri (C₁-C₄)alkylamino, monohydroxy(C₁- C₄) alkylamino, imidazolinium and ammonium radicals.

Among the para-phenylenediamines of formula (2) above, mention may be made more particularly of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-3-methylaniline, 4-amino-3-chloro-N,N-bis(β-hydroxyethyl)aniline, 2-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(βγ-dihydroxypropyl)-para-phenylenediamine, N-(4′-aminophenyl)-para-phenylenediamine, N-phenyl-para-phenylenediamine and 2β-hydroxyethyloxy-para-phenylenediamine, and the addition salts thereof with an acid.

Among the para-phenylenediamines of formula (2) above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine and 2-chloro-para-phenylenediamine, and the addition salts thereof with an acid are most particularly preferred.
(II) the double bases are compounds comprising at least two aromatic nuclei bearing amino and/or hydroxyl groups.

Among the double bases which can be used as oxidation bases in the dye compositions in accordance with the invention, mention may be made in particular of the compounds corresponding to formula (3) below, and the addition salts thereof with an acid: 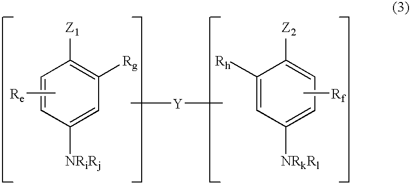
in which:
Z₁ and Z₂, which may be identical or different, represent a hydroxyl or —NH₂ radical which may be substituted with a C₁-C₄ alkyl radical or with a linker arm Y;
the linker arm Y represents a linear or branched alkylene chain containing from 1 to 14 carbon atoms, which may be interrupted by or terminated with one or more nitrogenous groups and/or one or more hetero atoms such as oxygen, sulphur or nitrogen atoms, and optionally substituted with one or more hydroxyl or C₁-C₆ alkoxy radicals;
Rₑ and R_{f} represent a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical or a linker arm Y;
R_{g}, Rₕ, Rᵢ, Rⱼ, Rₖ and Rₗ, which may be identical or different, represent a hydrogen atom, a linker arm Y or a C₁-C₄ alkyl radical; it being understood that the compounds of formula (3) contain only one linker arm Y per molecule.

Among the nitrogenous groups of formula (3) above, mention may be made in particular of amino, mono(C₁-C₄) alkylamino, di (C₁-C₄)alkylamino, tri (C₁-C₄)alkylamino, monohydroxy (C₁-C₄) alkylamino, imidazolinium and ammonium radicals.

Among the double bases of formula (3) above, mention may be made more particularly of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis-(ethyl)-N,N′-bis(4′-amino-3′-methylphenyl)ethylenediamine and 1,8-bis(2,5-diaminophenoxy)-3,6-dioxaoctane, and the addition salts thereof with an acid. Among these double bases of formula (3), N,N′-bis(β-hydroxyethyl)-N,N′-bis(4′-amino-phenyl) -1,3-diaminopropanol and 1,8-bis(2,5-diaminophenoxy)-3,6-dioxaoctane, or one of the addition salts thereof with an acid, are particularly preferred.
(III) the para-aminophenols corresponding to formula (4) below, and the addition salts thereof with an acid: 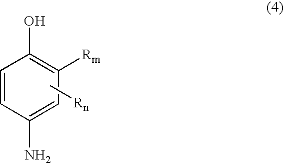
   in which:
Rₘ represents a hydrogen or halogen atom such as fluorine or a C₁-C₄ alkyl, C₁-C₄ monohydroxyalkyl, (C₁-C₄)alkoxy(Cl-C₄)-alkyl, C₁-C₄ aminoalkyl or hydroxy(C₁-C₄)alkylamino(C₁-C₄)alkyl radical,
Rₙ represents a hydrogen or halogen atom such as fluorine or a C₁-C₄-alkyl, C₁-C₄ monohydroxyalkyl, C₂-C4 polyhydroxyalkyl, C₁-C₄ aminoalkyl, C₁-C₄ cyanoalkyl or (C₁-C₄)alkoxy(Cl-C₄)alkyl radical.

Among the para-aminophenols of formula (4) above, mention may be made more particularly of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol and 4-amino-2-(□-hydroxyethylaminomethyl)phenol, and the addition salts thereof with an acid.
(IV) the ortho-aminophenols that may be used as oxidation bases in the context of the present invention are chosen especially from 2-aminophenol, 2-amino-1-hydroxy-5-methylbenzene, 2-amino-l-hydroxy-6-methylbenzene and 5-acetamido-2-aminophenol, and the addition salts thereof with an acid.
(V) among the heterocyclic bases which can be used as oxidation bases in the dye compositions in accordance with the invention, mention may be made more particularly of pyridine derivatives, pyrimidine derivatives and pyrazole derivatives and the addition salts thereof with an acid.

Among the pyridine derivatives, mention may be made more particularly of the compounds described, for example, in patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3amino-6-methoxypyridine and 3,4-diaminopyridine, and the addition salts thereof with an acid.

Among the pyrimidine derivatives, mention may be made more particularly of the compounds described, for example, in patents DE 2 359 399; JP 88-169 571; JP 91-10659 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048, and among which mention may be made of pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]-pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine and 2,5,N7,N7-tetramethylpyrazolo[1,5-a]pyrimidine-3,7-diamine, the addition salts thereof, and the tautomeric forms thereof, when a tautomeric equilibrium exists, and the addition salts thereof with an acid.

According to the present invention, the additional oxidation bases may preferably represent from 0.0005% to 12% by weight approximately relative to the total weight of the dye composition.

In general, the addition salts with an acid which can be used in the context of the dye compositions of the invention (oxidation bases and couplers) are chosen in particular from the hydrochlorides, hydrobromides, sulphates, tartrates, lactates and acetates.

The medium that is suitable for the dyeing (or the support) generally consists of water or of a mixture of water and at least one organic solvent in order to dissolve the compounds that would not be sufficiently soluble in water. By way of organic solvent, mention may be made, for example, of C₁-C₄ lower alkanols such as ethanol and isopropanol; glycerol; glycols and glycol ethers such as 2-butoxyethanol, propylene glycol, propylene glycol monomethyl ether, diethylene glycol monoethyl ether and monomethyl ether, and aromatic alcohols such as benzyl alcohol or phenoxyethanol, similar products and mixtures thereof.

The solvents can be present in proportions preferably of between 1 and 40% by weight approximately relative to the total weight of the dye composition, and even more preferably between 5 and 30% by weight approximately.

The pH of the dye composition in accordance with the invention is generally between 3 and 12 approximately and preferably between 5 and 11 approximately. It may be adjusted to the desired value with the aid of acidifying or basifying agents commonly used in the dyeing of keratin fibres.

Among the acidifying agents, mention may be made, by way of example, of inorganic or organic acids such as hydrochloric acid, orthophosphoric acid, carboxylic acids such as tartaric acid, citric acid or lactic acid, and sulphonic acids.

Among the basifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds of formula (5) below: 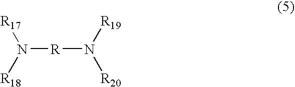
in which R is a propylene residue optionally substituted with a hydroxyl group or a C₁-C₄ alkyl radical; R₁₇, R₁₈, R₁₉ and R₂₀, which may be identical or different, represent a hydrogen atom or a C₁-C₄ alkyl or C₁-C₄ hydroxyalkyl radical.

The dye composition in accordance with the invention can also contain various adjuvants used conventionally in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, antioxidants, penetration agents, sequestering agents, fragrances, buffers, dispersing agents, conditioners, such as, for example, volatile or non-volatile silicones, which are modified or unmodified, film-forming agents, ceramides, preserving agents and opacifiers.

Needless to say, a person skilled in the art will take care to select this or these optional complementary compound(s) such that the advantageous properties intrinsically associated with the combination in accordance with the invention is (are) not, or not substantially, adversely affected by the addition or additions envisaged.

The dye composition in accordance with the invention can be in various forms, such as in the form of liquids, creams or gels or in any other form that is suitable for dyeing keratin fibres, and especially human hair.

A subject of the invention is also a process for dyeing keratin fibres, and in particular human keratin fibres such as the hair, using the dye composition as defined above.

According to this process, the dye composition as defined above is applied to the fibres, the colour being developed at acidic, neutral or alkaline pH using an oxidizing agent, this oxidizing agent possibly being added just at the time of use to the dye composition or by means of an oxidizing composition applied simultaneously or sequentially.

According to one particularly preferred embodiment of the dyeing process according to the invention, the dye composition described above is mixed, at the time of use, with an oxidizing composition containing, in a medium which is suitable for dyeing, at least one oxidizing agent present in an amount which is sufficient to develop a coloration. The mixture obtained is then applied to the keratin fibres and is left to stand on them for about 3 to 60 minutes, preferably about 5 to 40 minutes, after which the fibres are rinsed, washed with shampoo, rinsed again and dried.

The oxidizing agent present in the oxidizing composition as defined above may be chosen from the oxidizing agents conventionally used for the oxidation dyeing of keratin fibres, and among which mention may be made of hydrogen peroxide, urea peroxide, alkali metal bromates, persalts such as perborates, percarbonates and persulphates, and peracids. Hydrogen peroxide is particularly preferred.

The pH of the oxidizing composition containing the oxidizing agent as defined above is such that, after mixing it with the dye composition, the pH of the resulting composition applied to the keratin fibres preferably ranges between about 3 and 12 and even more preferably between 5 and 11. It is adjusted to the desired value by means of acidifying or basifying agents usually used for the dyeing of keratin fibres and as defined above.

The oxidizing composition as defined above can also contain various adjuvants conventionally used in compositions for dyeing the hair and as defined above.

The dye composition that is applied to the keratin fibres can be in various forms, such as in the form of liquids, creams or gels or any other form that is suitable for dyeing keratin fibres, and in particular human hair.

According to one variant, a composition containing at least the cellulose-based compound is applied to these fibres in a first stage, and a composition containing at least one diaminopyrazole is applied in a second stage, the application of the composition containing the cellulose-based compound(s) possibly being followed by a rinsing step, the colour being developed using an oxidizing agent.

Another subject of the invention is a multi-compartment device or dyeing “kit” or any other multi-compartment packaging system, a first compartment of which contains the dye composition as defined above and a second compartment of which contains the oxidizing composition as defined above. These devices may be equipped with a means for applying the desired mixture to the hair, such as the devices described in patent FR-2 586 913 in the name of the Applicant.

According to a different embodiment, the device comprises at least three compartments, a first compartment that contains the cellulose-based compound that is useful for the invention, a second compartment that contains a diaminopyrazole, and a third compartment that contains an oxidizing composition.

The examples that follow are intended to illustrate the invention without, however, limiting its scope.

### EXAMPLES

### Dyeing Examples 1 to 4

The dye compositions below, in accordance with the invention, were prepared (amounts in grams):

| | EXAMPLES | | | |
|---|---|---|---|---|
| | 1 | 2 | 3 | 4 |
| 4,5-Diamino-1β-hydroxyethyl- | 0.645 | 0.645 | 0.645 | 0.645 |
| pyrazole dihydrochloride | | | | |
| (oxidation base) | | | | |
| 3-Amino-6-methylphenol | 0.369 | 0.369 | 0.369 | 0.369 |
| (coupler) | | | | |
| Cellulose powder sold under | 0.1 | — | — | — |
| the name Cepo S/100 by the | | | | |
| company Svenska (cellulose- | | | | |
| based compound according to | | | | |
| the invention) | | | | |
| Hydroxyethylcellulose sold- | — | 0.1 | — | — |
| under the name Natrosol 250 MR | | | | |
| by the company Aqualon | | | | |
| (cellulose-based compound | | | | |
| according to the invention) | | | | |
| Carboxymethylcellulose, sodium | — | — | 0.15 | — |
| salt, sold under the name | | | | |
| Blanose 7M8/SF by the company | | | | |
| Aqualon (cellulose-based | | | | |
| compound according to the | | | | |
| invention) | | | | |
| Hydroxyethylcellulose | — | — | — | 0.2 |
| quaternized with | | | | |
| glycidyltrimethylammonium | | | | |
| chloride, sold under the name | | | | |
| Natrosol ADX 237 by the | | | | |
| company Hercules (cellulose- | | | | |
| based compound according to | | | | |
| the invention) | | | | |
| Common dye support | (**) | (**) | (**) | (**) |
| Demineralized water qs | 100 g | 100 g | 100 g | 100 g |

(**) common dye support containing (AM meaning active material):

| | | |
|---|---|---|
| Oleyl alcohol polyglycerolated with | 4.0 | g |
| 2 mol of glycerol | | |
| Oleyl alcohol polyglycerolated with | 5.69 | g A.M. |
| 4 mol of glycerol, containing 78% | | |
| active material (A.M.) | | |
| Oleic acid | 3.0 | g |
| Oleylamine containing 2 mol of | 7.0 | g |
| ethylene oxide, sold under the trade | | |
| name Ethomeen O12 by the company Akzo | | |
| Diethylaminopropyl laurylamino- | 3.0 | g A.M. |
| succinamate, sodium salt, containing | | |
| 55% A.M. | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9.0 | g |
| Sodium metabisulphite as an aqueous | 0.455 | g A.M. |
| solution containing 35% A.M. | | |
| Ammonium acetate | 0.8 | g |
| Antioxidant, sequestering agent | qs | |
| Fragrance, preserving agent | qs | |
| Aqueous ammonia containing 20% NH₃ | 10 | g |

At the time of use, each dye composition above was mixed with an equal amount by weight of an oxidizing composition consisting of a 20-volumes aqueous hydrogen peroxide solution (6% by weight).

Each resulting composition was applied for 30 minutes to locks of natural grey hair containing 90% white hairs. The locks of hair were then rinsed, washed with a standard shampoo and then dried.

In all cases, a strong and resistant red shade, with good harmlessness, is obtained.

### Dyeing Examples 5 to 7

The dye compositions below, in accordance with the invention, were prepared (amounts in grams):

| | EXAMPLE | | |
|---|---|---|---|
| | 5 | 6 | 7 |
| 4,5-Diamino-1-ethyl-3-methyl- | 0.639 | 0.639 | — |
| pyrazole dihydrochloride (oxidation | | | |
| base) | | | |
| 3,4,5-Triaminopyrazole dihydro- | — | — | 0.667 |
| chloride (oxidation base) | | | |
| 3-Amino-6-methylphenol (coupler) | 0.369 | 0.369 | 0.369 |
| Microcrystalline cellulose sold | 0.1 | — | 0.1 |
| under the name Avicel PH101 by the | | | |
| company FMC (cellulose-based | | | |
| compound according to the | | | |
| invention) | | | |
| Hydroxyethylcellulose sold under | — | 0.25 | — |
| the name Natrosol 250 MR by the | | | |
| company Aqualon (cellulose-based | | | |
| compound according to the | | | |
| invention) | | | |
| Common dye support | (**) | (**) | (**) |
| Demineralized water qs | 100 g | 100 g | 100 g |

(**) common dye support: identical to that of Examples 1 to 4.

(**) common dye support: identical to that of Examples 1 to 4.

At the time of use, each dye composition above was mixed with an equal amount by weight of an oxidizing composition consisting of a 20-volumes aqueous hydrogen peroxide solution (6% by weight).

Each resulting composition was applied for 30 minutes to locks of natural grey hair containing 90% white hairs. The locks of hair were then rinsed, washed with a standard shampoo and then dried.

In the three cases, a strong and resistant shade, with good harmlessness, is obtained.

1. Composition for the oxidation dyeing of human keratin fibres, and in particular of human keratin fibres such as the hair, comprising, in a medium that is suitable for dyeing:
at least one oxidation base chosen from 4,5-diamino-1-hydroxytethylpyrazole and triaminopyrazoles,
at least one cellulose-based compound chosen from unsubstituted celluloses and cellulose ethers.

2. Composition according to claim 1, characterized in that the nonionic cellulose ethers are chosen from alkylcelluloses, hydroxyalkylcelluloses and hydroxyalkyl-alkylcelluloses.

3. Composition according to claim 1 or 2, characterized in that the hydroxyalkylcelluloses are chosen from hydroxymethylcelluloses, hydroxyethyl-celluloses and hydroxypropylcelluloses.

4. Composition for the oxidation dyeing of human keratin fibres, and in particular of human keratin fibres such as the hair, characterized in that it comprises, in a medium that is suitable for dyeing:
at least one oxidation base chosen from 4,5-diamino-1-hydroxyethylpyrazole and triaminopyrazoles, and
at least one cellulose-based compound chosen from unsubstituted celluloses; anionic or cationic cellulose ethers; nonionic cellulose ethers chosen from alkylcelluloses, hydroxymethylcelluloses, hydroxypropylcelluloses and hydroxyalkylalkylcelluloses.

5. Composition according to claim 2 or 4, characterized in that the alkylcelluloses are chosen from methylcelluloses and ethylcelluloses.

6. Composition according to claim 2 or 4, characterized in that the hydroxyalkyl-alkylcelluloses are chosen from hydroxypropyl-methylcelluloses, hydroxyethyl-methylcelluloses, hydroxyethyl-ethylcelluloses and hydroxybutyl-methylcelluloses.

7. Composition according to claim 1 or 4, characterized in that the anionic cellulose ethers are chosen from carboxyalkylcelluloses and salts thereof.

8. Composition according to claim 7, characterized in that the carboxyalkylcelluloses are chosen from carboxymethylcelluloses, carboxymethyl-methylcelluloses and carboxymethylhydroxyethyl-celluloses, and the sodium salts thereof.

9. Composition according to claim 1 or 4, characterized in that the cationic cellulose ethers are chosen from crosslinked or non-crosslinked quaternized hydroxyethylcelluloses.

10. Composition according claims 1 or 4, characterized in that the cellulose-based compound(s) represent(s) from 0.00001% to 10% and preferably from 0.001% to 5% by weight approximately relative to the total weight of the dye composition, and even more preferably from 0.001% to 3% by weight approximately relative to this weight.

11. Composition according to claims 1 or 4, characterized in that the triaminopyrazoles are chosen from the compounds of formula (III) below, and/or the addition salts thereof with an acid: 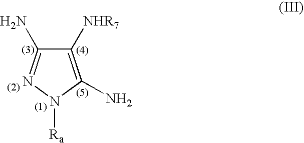
in which:
R₇ and R₈, which may be identical or different, represent a hydrogen atom or a C₁-C₄ alkyl or C₂-C₄ hydroxyalkyl radical.

12. Composition according to claim 11, characterized in that the triaminopyrazoles of formula (III) are chosen from 3,4,5-triaminopyrazole, 1-methyl 3,4,5-triaminopyrazole, 3,5-diamino-1-methyl-4-methyl aminopyrazole and 3,5-diamino-4-(β-hydroxyethyl)amino 1-methylpyrazole, and the addition salts thereof with an acid.

13. Composition according to claims 1 or 4, characterized in that it contains as oxidation base 4,5-diamino-1-hydroxyethylpyrazole or an addition salt thereof with an acid.

14. Composition for the oxidation dyeing of human keratin fibres, and in particular of human keratin fibres such as the hair, characterized in that it comprises, in a medium that is suitable for dyeing:
as oxidation base, at least 4,5-diamino-1-hydroxy-ethylpyrazole or an addition salt thereof with an acid; and
at least one hydroxyethylcellulose.

15. Composition according to claims 1, 4 or 14, characterized in that the 4,5-diamino-1-hydroxyethylpyrazole and/or the triaminopyrazole(s) and/or the corresponding addition salt(s) with an acid represent from 0.0005% to 12% by weight relative to the total weight of the dye composition.

16. Composition according to claim 15, characterized in that the 4,5-diamino-1-hydroxyethylpyrazole and/or the triaminopyrazole(s) and/or the corresponding addition salt(s) with an acid represent from 0.005% to 6% by weight relative to the total weight of the dye composition.

17. Composition according to claim 1, 4 or 14, characterized in that the weight ratio of the cellulose-based compound(s) to the 4,5-diamino-1-hydroxyethylpyrazole and/or the triaminopyrazole(s) and/or the addition salt(s) with an acid is between 0.001 and 100 and preferably between 0.01 and 10.

18. Composition according to claims 1, 4 or 14, characterized in that the addition salts with an acid are chosen from the hydrochlorides, hydrobromides, sulphates, tartrates, lactates and acetates.

19. Composition according to claims 1, 4 or 14, characterized in that it contains at least one coupler.

20. Composition according to claim 14, characterized in that the coupler(s) represent(s) from 0.0001% to 10% by weight approximately relative to the total weight of the dye composition, and even more preferably from 0.005% to 5% by weight approximately relative to this weight.

21. Composition according to claims 1, 4 or 14, characterized in that it contains at least one additional oxidation base other than the 4,5-diamino-1-hydroxyethylpyrazole or triaminopyrazoles.

22. Composition according to claim 21, characterized in that the additional oxidation base(s) represent(s) from 0.0005% to 12% by weight approximately relative to the total weight of the dye composition.

23. Composition according to claims 1, 4 or 14, characterized in that it has a pH of between 3 and 12.

24. Composition according to claims 1, 4 or 14, characterized in that it is in the form of liquids, creams or gels or in any other form that is suitable for dyeing keratin fibres, and especially human hair.

25. Process for dyeing keratin fibres, and in particular human keratin fibres such as the hair, characterized in that at least one dye composition as defined in claims 1, 4 or 14 is applied to these fibres, and the colour is revealed at acidic, neutral or alkaline pH using an oxidizing agent.

26. Process according to claim 25, characterized in that the oxidizing agent present in the oxidizing composition is chosen from hydrogen peroxide, urea peroxide, alkali metal bromates, persalts such as perborates, percarbonates and persulphates, and peracids.

27. Process for dyeing keratin fibres, and in particular human keratin fibres such as the hair, characterized in that a composition containing at least one cellulose-based compound as defined in claims 1, 4 or 14 is applied to these fibres in a first stage, a composition containing at least one diaminopyrazole as defined in claims 1, 4 or 14 is applied in a second stage, the application of the composition containing the cellulose-based compound(s)possibly being followed by a rinsing step, the colour being developed using an oxidizing agent.

28. Multi-compartment device comprising a first compartment containing a dye composition as defined in claims 1, 4 or 14 and a second compartment containing an oxidizing composition.

29. Multi-compartment device comprising a first compartment containing a cellulose-based compound as defined in claims 1, 4 or 14, a second compartment containing a diaminopyrazole as defined in claims 1, 4 or 14, and a third compartment containing an oxidizing composition.
